(19) United States
(12) Patent Application Publication
(10) Pub. No.: US 2008/0076780 A1
(43) Pub. Date: Mar. 27, 2008

(54) Combination of N-(3-Methoxy-5-Methylpyrazin-2-Yl)-2-(4-[1,3,4-Oxadiazol-2-Yl]Phenyl)Pyridine-3-Sulphonamide and an Anti-Mitotic Cytotoxic Agent

(75) Inventors: John Curwen, Cheshire (GB); Andrew Hughes, Cheshire (GB); Donna Johnstone, Cheshire (GB); Francis Thomas Boyle, Cheshire (GB); Gwen Boyle, Congleton (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 11/720,001

(22) PCT Filed: Nov. 23, 2005
(86) PCT No.: PCT/GB05/04483

§ 371 Date: Sep. 12, 2007

(30) Foreign Application Priority Data
Nov. 25, 2004 (GB) 0425854.7

(51) Int. Cl. A61K 31/497; A61P 35/00

(52) U.S. Cl. 514/255.05

(57) ABSTRACT

A combination, comprising N-(3-methoxy-5-methylpyrazin-2-yl)-2-(4-[1,3,4-oxadiazol-2-yl]phenyl)pyridine-3-sulphonamide and an anti-mitotic cytotoxic agent.

The present invention relates to combinations comprising N-(3-methoxy-5-methylpyrazin-2-yl)-2-(4-[1,3,4-oxadiazol-2-yl]phenyl)pyridine-3-sulphonamide, or a pharmaceutically acceptable salt thereof, hereafter “Compound (I)”, and an anti-mitotic cytotoxic agent. These combinations are useful for the treatment or prophylaxis of cancer. The invention also relates to a pharmaceutical composition comprising such combinations and to the use thereof in the manufacture of a medicament for use in the treatment or prophylaxis of cancer, in particular prostate cancer.

Cancer affects an estimated 10 million people worldwide. This figure includes incidence, prevalence and mortality. More than 4.4 million cancer cases are reported from Asia, including 2.5 million cases from Eastern Asia, which has the highest rate of incidence in the world. By comparison, Europe has 2.8 million cases, North America 1.4 million cases, and Africa 627,000 cases.

In the UK and US, for example, more than one in three people will develop cancer at some point in their life. Cancer mortality in the U.S. is estimated to account for about 600,000 a year, about one in every four deaths, second only to heart disease in percent of all deaths, and second to accidents as a cause of death of children 1-14 years of age. The estimated cancer incidence in the U.S. is now about 1,380,000 new cases annually, exclusive of about 900,000 cases of non-melanotic (basal and squamous cell) skin cancer.

Cancer is also a major cause of morbidity in the UK with nearly 260,000 new cases (excluding non-melanoma skin cancer) registered in 1997. Cancer is a disease that affects mainly older people, with 65% of cases occurring in those over 65. Since the average life expectancy in the UK has almost doubled since the mid nineteenth century, the population at risk of cancer has grown. Death rates from other causes of death, such as heart disease, have fallen in recent years while deaths from cancer have remained relatively stable. The result is that 1 in 3 people will be diagnosed with cancer during their lifetime and 1 in 4 people will die from cancer. In people under the age of 75, deaths from cancer outnumber deaths from diseases of the circulatory system, including ischaemic heart disease and stroke. In 2000, there were 151,200 deaths from cancer. Over one fifth (22 percent) of these were from lung cancer, and a quarter (26 percent) from cancers of the large bowel, breast and prostate.

Worldwide, the incidence and mortality rates of certain types of cancer (of stomach, breast, prostate, skin, and so on) have wide geographical differences which are attributed to racial, cultural, and especially environmental influences. There are over 200 different types of cancer but the four major types, lung, breast, prostate and colorectal, account for over half of all cases diagnosed in the UK and US. Prostate cancer is the fourth most common malignancy among men worldwide, with an estimated 400,000 new cases diagnosed annually, accounting for 3.9 percent of all new cancer cases.

Current options for treating cancers include surgical resection, radiation therapy and/or systemic chemotherapy. These are partially successful in some forms of cancer, but are not successful in others. There is a clear need for new therapeutic treatments.

Recently, endothelin A receptor antagonists have been identified as potentially of value in the treatment of cancer (Cancer Research, 56, 663-668, Feb. 15, 1996 and Nature Medicine, Volume 1, Number 9, September 1999, 944-949).

The endothelins are a family of endogenous 21 amino acid peptides comprising three isoforms, endothelin-1, endothelin-2 and endothelin-3. The endothelins are formed by cleavage of the Trp²¹-Val²² bond of their corresponding proendothelins by an endothelin converting enzyme. The endothelins are among the most potent vasoconstrictors known. They exhibit a wide range of other activities including stimulation of cell proliferation and mitogenesis, inhibition of apoptosis, extravasation and chemotaxis, and also interact with a number of other vasoactive agents.

The endothelins are released from a range of tissue and cell sources including vascular endothelium, vascular smooth muscle, kidney, liver, uterus, airways, intestine and leukocytes. Release can be stimulated by hypoxia, shear stress, physical injury and a wide range of hormones and cytokines. Elevated endothelin levels have been found in a number of disease states in man including cancers.

Compound (I) is a specific endothelin A antagonist, a property which makes it particularly suitable for the treatment of cancers (see WO 2004/018044).

Anti-mitotic cytotoxic agents that bind to tubulin (a protein involved closely in cell division and therefore in multiplication of cancer cells and tumour growth), inhibit mammalian cell growth by interfering with cell division. At a molecular level they can either cause stabilisation (epothilones and taxanes) or destabilisation (vinca alkaloids) of the microtubules involved in chromosome segregation during mitosis. Cells treated with these drugs are held in mitosis, i.e. they interfere with the cell division process, this may eventually result in cell death due to unsuccessful mitosis.

The present inventors have unexpectedly found that the combination of Compound (I) and an anti-mitotic cytotoxic agent can have a particular beneficial and/or synergistic effect in the treatment of cancer.

Therefore according to the present invention, there is provided a combination, comprising Compound (I) and an anti-mitotic cytotoxic agent.

Herein where the term “anti-mitotic cytotoxic agent” is used it is to be understood that this refers to any chemical analogue which exerts its anticancer effect by stabilization or destabilisation of the tubulin microtubules involved in cell division.

Examples of “anti-mitotic cytotoxic agents” include taxanes, epothilones and vinca alkaloids. Particular examples of “anti-mitotic cytotoxic agents” are:
TAXANES: such as (2aR,3aR,4aR,6R,9S,11S,12S,12aR,12bS)-6,12b-diacetoxy-9-[3(S)-(tert-butoxycarbonylamino)-2(R)-hydroxy-3-phenylpropionyloxy]-12-benzoyloxy-1′-hydroxy-8,13,13-trimethyl-2a,3,3a,4,5,6,9,10,11,12,12a,12b-dodecahydro-1H-7,11-methanocyclodeca[3,4]-cyclopropa[4,5]benz[1,2-b]oxet-5-one dihydrate; Paclitaxel (Taxol), BMS184476 (7-methylthiomethylpaclitaxel); BMS 188797; BMS 275183; CYC-3204 (a penetratin-paclitaxel conjugate); Taxoprexin; DJ-927; Docetaxel (Taxotere); XRP9881 (RPR-109881A); XRP6258 (RPR112658); Milataxel; MST 997; MBT-206; NBT-287; ortataxel; Protax-3; PG-TXL; PNU-166945; 106258; BMS-188797; 109881; BAY 598862 (IDN 5109; semisynthetic taxane); Protaxel and MAC-321 (Taxalog);
EPOTHILONES: derivatives and analogues of:
   epothilone A;
   epothilone B such as: ABJ879; BMS247550 (ixabepilone); EP0906 (patupilone); ZK EPO;
   epothilone C; and
   epothilone D such as: KOS 862;
VINCA ALKALOIDS ANALOGUES AND DERIVATIVES: vincristine; vinblastine; vinorelbine; vinflunine; Rhizoxin
OTHER TUBULIN ANTAGONISTS:
   beta-tubulin binders/antagonists such as: T-138067; T 900607; D 24851; STA 5312
   anti-microtubule agents such as: HTI-286 (hemiasterlin derivative) Dolastatin derivatives (ILX-651); halichondrin analogues such as E7389; cryptophycin analogues; and discodermolides (NVP-XAA296).

In one aspect, the present invention relates the combination of Compound (I) and any one of the above compounds.

In a further aspect of the invention there is provided Compound (I) and a taxane.

In a further aspect of the invention there is provided Compound (I) and an epothilone.

In a further aspect of the invention there is provided Compound (I) and an epothilone A derivative or analogue thereof.

In a further aspect of the invention there is provided Compound (I) and an epothilone B derivative or analogue thereof.

In a further aspect of the invention there is provided Compound (I) and an epothilone C derivative or analogue thereof.

In a further aspect of the invention there is provided Compound (I) and an epothilone D derivative or analogue thereof.

In a further aspect of the invention there is provided Compound (I) and a vinca alkaloid derivative or analogue thereof.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial and/or synergistic effect of the combination.

In one aspect, where a compound or a pharmaceutically acceptable salt thereof, is referred to this refers to the compound only. In another aspect this refers to a pharmaceutically acceptable salt of the compound.

Where cancer is referred to, particularly it refers to oesophageal cancer, myeloma, hepatocellular, pancreatic, cervical cancer, ewings tumour, neuroblastoma, kaposis sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, brain cancer, renal cancer, lymphoma and leukaemia. More particularly it refers to prostate cancer. In addition, more particularly it refers to SCLC, NSCLC, colorectal cancer, ovarian cancer and/or breast cancer. In addition, more particularly it refers to SCLC. In addition, more particularly it refers to NSCLC. In addition, more particularly it refers to colorectal cancer. In addition, more particularly it refers to ovarian cancer. In addition, more particularly it refers to breast cancer. Furthermore, more particularly it refers to bladder cancer, oesophageal cancer, gastric cancer, melanoma, cervical cancer and/or renal cancer. In addition it refers to endometrial, liver, stomach, thyroid, rectal and/or brain cancer. In another aspect of the invention, the cancer is not melanoma. In another embodiment of the invention, particularly the cancer is in a metastatic state, and more particularly the cancer produces metastases to the bone. In a further embodiment of the invention, particularly the cancer is in a metastatic state, and more particularly the cancer produces skin metastases. In a further embodiment of the invention, particularly the cancer is in a metastatic state, and more particularly the cancer produces lymphatic metastases. In a further embodiment of the invention, the cancer is in a non-metastatic state.

Where the treatment of cancer is referred to particularly this is the treatment of cancerous tumours expressing endothelin A. This treatment is in terms of one or more of the extent of the response, the response rate, the time to disease progression and the survival rate.

Particular combinations of the present invention include:

Compound (I) and paclitaxel;

Compound (I) and docetaxel;

Compound (I) and ixabepilone;

Compound (I) and patupilone;

Compound (I) and vinorelbine;

Compound (I) and XAA296; and

Compound (I) and T-138067.

Suitable pharmaceutically-acceptable salts include, for example, salts with alkali metal (such as sodium, potassium or lithium), alkaline earth metals (such as calcium or magnesium), ammonium salts, and salts with organic bases affording physiologically acceptable cations, such as salts with methylamine, dimethylamine, trimethylamine, piperidine and morpholine. In addition, for those compounds which are sufficiently basic, suitable pharmaceutically-acceptable salts include, pharmaceutically-acceptable acid-addition salts with hydrogen halides, sulphuric acid, phosphoric acid and with organic acids such as citric acid, maleic acid, methanesulphonic acid and p-toluenesulphonic acid. Alternatively, the compounds may exist in zwitterionic form.

Therefore according to the present invention, there is provided a combination, comprising Compound (I) and an anti-mitotic cytotoxic agent for use as a medicament.

According to a further aspect of the invention there is provided a pharmaceutical composition which comprises Compound (I) and an anti-mitotic cytotoxic agent in association with a pharmaceutically acceptable diluent or carrier.

According to a further aspect of the invention there is provided a pharmaceutical composition which comprises Compound (I), in association with a pharmaceutically acceptable diluent or carrier, in combination with a pharmaceutical composition which comprises an anti-mitotic cytotoxic agent in association with a pharmaceutically acceptable diluent or carrier.

Therefore according to the present invention, there is provided a method of treating cancer, in a warm-blooded animal, such as man, in need of such treatment which comprises administering to said animal an effective amount of Compound (I) in combination with an effective amount of an anti-mitotic cytotoxic agent.

For the avoidance of doubt, where the treatment of cancer is indicated, it is to be understood that this also refers to the prevention of metastases and the treatment of metastases, i.e. cancer spread. Therefore the combination of the present invention could be used to treat a patient who has no metastases to stop them occurring, or to lengthen the time period before they occur, and to a patient who already has metastases to treat the metastases themselves. Furthermore the treatment of cancer also refers to treatment of an established primary tumour or tumours and developing primary tumour or tumours. In one aspect of the invention the treatment of cancer relates to the prevention of metastases. In another aspect of the invention the treatment of cancer relates to the treatment of metastases. In another aspect of the invention the treatment of cancer relates to treatment of an established primary tumour or tumours or developing primary tumour or tumours. Herein, the treatment of cancer also refers to the prevention of cancer per se.

In addition the treatment of cancer also refers to the production of an anti-angiogenic effect in a warm blooded animal.

In addition the treatment of cancer also refers to the production of an anti-proliferative effect in a warm blooded animal.

According to a further aspect of the present invention there is provided a kit comprising Compound (I) and an anti-mitotic cytotoxic agent; optionally with instructions for use.

According to a further aspect of the present invention there is provided a kit comprising:

a) Compound (I), in a first unit dosage form;

b) an anti-mitotic cytotoxic agent; in a second unit dosage form; and

c) container means for containing said first and second dosage forms; and optionally

d) with instructions for use.

An example of a unit dosage from for Compound (I) might be a tablet for oral formulation, see that described herein below. For an example of a unit dosage from for an anti-mitotic cytotoxic agent see herein below.

According to a further aspect of the present invention there is provided a kit comprising:

a) Compound (I), together with a pharmaceutically acceptable diluent or carrier, in a first unit dosage form;

b) an anti-mitotic cytotoxic agent, in a second unit dosage form; and

c) container means for containing said first and second dosage forms; and optionally

d) with instructions for use.

According to a further aspect of the invention there is provided a pharmaceutical composition which comprises Compound (I) and an anti-mitotic cytotoxic agent in association with a pharmaceutically acceptable diluent or carrier for use in the treatment of cancer.

According to a further aspect of the invention there is provided a pharmaceutical composition which comprises Compound (I), in association with a pharmaceutically acceptable diluent or carrier, in combination with a pharmaceutical composition which comprises an anti-mitotic cytotoxic agent in association with a pharmaceutically acceptable diluent or carrier for use in the treatment of cancer.

The pharmaceutical compositions may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository. In general the above compositions may be prepared in a conventional manner using conventional excipients.

For example Compound (I) can be formulated as a tablet using the following excipients:
Compound (I);
Lactose monohydrate (filler);
Croscarmellose sodium (disintegrant);
Povidone (binder);
Magnesium stearate (lubricant);
Hypromellose (film coat component);
Polyethylene glycol 300 (film coat component); and
Titanium dioxide (film coat component).

Anti-mitotic cytotoxic agents may be formulated according to known procedures. For example various formulations of Paclitaxel are known. These include Abraxane; Acusphere; AI-850; DO/NDR/02 (a cremophor-free paclitaxel formulation); EndoTag-1; liposome encapsulated paclitaxel; LPE/PLP Paclitaxel; MPI-5019; NK-105; OncoGel; Paclimer Microspheres; S-8184; ABI-007; NOVA-12005; SP-1010C-O; Pacligel; SP-1010C; Paxoral, Xorane; Genexol; Tocosol; PacoExtra; Yewtaxan; Taxosomes; Atrigel; Xyotax (paclitaxel polyglumex; polyglutamated paclitaxel) and SP 1010C.

According to a further aspect of the present invention there is provided a kit comprising Compound (I) and an anti-mitotic cytotoxic agent; optionally with instructions for use; for use in the treatment of cancer.

According to a further aspect of the present invention there is provided a kit comprising:

a) Compound (I), in a first unit dosage form;

b) an anti-mitotic cytotoxic agent in a second unit dosage form; and

c) container means for containing said first and second dosage forms; and optionally

d) with instructions for use;

for use in the treatment of cancer.

According to a further aspect of the present invention there is provided a kit comprising:

a) Compound (I), together with a pharmaceutically acceptable diluent or carrier, in a first unit dosage form;

b) an anti-mitotic cytotoxic agent in a second unit dosage form; and

c) container means for containing said first and second dosage forms; and optionally

d) with instructions for use;

for use in the treatment of cancer.

According to another feature of the invention there is provided the use of Compound (I), in combination with an anti-mitotic cytotoxic agent in the manufacture of a medicament for use in the treatment of cancer, in a warm-blooded animal, such as man.

According to another feature of the invention there is provided the use of Compound (I), in combination with an anti-mitotic cytotoxic agent in the treatment of cancer, in a warm-blooded animal, such as man.

According to a further aspect of the present invention there is provided a combination comprising Compound (I) and an anti-mitotic cytotoxic agent for use in the treatment of cancer.

According to a further aspect of the present invention there is provided a combination treatment comprising the administration of an effective amount of Compound (I), optionally together with a pharmaceutically acceptable diluent or carrier, in combination with an effective amount of an anti-mitotic cytotoxic agent optionally together with a pharmaceutically acceptable diluent or carrier to a warm-blooded animal, such as man in need of such therapeutic treatment for use in the treatment of cancer.

The amount of Compound (I), or a pharmaceutically acceptable salt thereof, administered would be that sufficient to provide the desired pharmaceutical effect. For instance, Compound (I) could be administered to a warm-blooded animal orally, at a unit dose less than 1 g daily but more than 2.5 mg. Particularly Compound (I) could be administered to a warm-blooded animal, at a unit dose of less than 250 mg per day. In another aspect of the invention, Compound (I) could be administered to a warm-blooded animal, at a unit dose of less than 130 mg per day. In a further aspect of the invention, Compound (I) could be administered to a warm-blooded animal, at a unit dose of less than 50 mg per day.

Anti-mitotic cytotoxic agents may be administered in amounts in accordance with approval guidelines. They are both species and schedule dependent with respect to their maximum tolerated dose.

The dosage of each of the drugs and their proportions have to be composed so that the best possible treatment effects, as defined by national and international guidelines (which are periodically reviewed and re-defined), will be met.

### LEGENDS TO FIGURES

FIG. 1 depicts a bar chart showing the effects of Compound (I), and Paclitaxel, either alone or in combination, on apoptosis in ovarian cancer cell lines HEY and OVCA 433.

FIG. 2 depicts a bar chart showing the effects of Compound (I) and Paclitaxel, either alone or in combination, on the growth of HEY ovarian carcinoma cells in vivo.

FIG. 3 depicts a bar chart showing the effects of increasing doses of two cytotoxics (paclitaxel and docetaxel), either alone or in combination with endothelin 1 or endothelin 1+Compound (I) on the numbers of viable prostate cells (PPC-1) in an in vitro culture system (increasing absorbance values reflects increased numbers of living cells).

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

### EXAMPLES

### Experiments Demonstrating Enhanced Activity of Compound (I) in Combination with Anti-Mitotic Cytotoxic Agents (Paclitaxel and Docetaxel)

Introduction

To evaluate the effect of Compound (I) in combination with an anti-mitotic cytotoxic agent (paclitaxel) on the growth of various carcinoma cells, we utilised two established human ovarian cell lines (HEY and OVCA 433**) which express functional endothelin A (ETa) receptors and secrete high levels of endothelin-1 (ET-1). ET-1 is an anti-apoptotic factor in many cell types, having this effect via ETa receptors.
**OVCA 433 was established from ascites obtained from a patient with advanced serous ovarian adenocarcinoma (Tsa, S W et al., (1995) Exp. Cell Res. 218: 499-507) and HEY was derived from a xenograft of a peritoneal deposit of a cystadenocarcinoma of the ovary (Buick, R. N. et al., (1985) Cancer Research 45: 3668-3676). PPC-1 cells were originally derived from a human prostate tumour and were obtained from the laboratory of Dr. J. Nelson, University of Pittsburgh).

Materials and Methods

In vitro studies in ovarian cells: Human ovarian tumour cell lines (OVCA 433 and HEY) were maintained in culture media containing serum until sufficient numbers were available for experimentation. At this time the cells were transferred into media without serum. After 24 hours of serum starvation, cells were treated with either Compound (I) (1 μM) or paclitaxel (60 nM), or Compound (I)+paclitaxel. Following treatment for 24 hours, apoptosis was measured by a standard cell detection ELISA Plus kit (Boehringer Manheim).

In vitro studies in prostate cells: Human prostate tumour cells (PPC-1) were maintained in culture medium containing serum until sufficient numbers were available for experimentation. After this time cells were transferred into media without serum. After 23 hours of serum starvation cells were treated with either endothelin 1 (10⁻⁷M) or endothelin 1+Compound (I) at 10⁻⁷M. An additional group of cells received vehicle control alone. One hour later cells were treated with paclitaxel or docetaxel at either 10⁻⁶M, 10⁻⁸M or 10⁻¹⁰M for 24 hours. At the end of this 24 hour period, viable cell numbers were measured by a standard MTT assay (Mossman, J Immunol Methods. (1983) 65, 55-63).

In vivo studies: Athymic mice were given subcutaneous injections of 1.5×10⁶ HEY cells into the flank. After 7 days, when established tumours had formed, mice were randomized to 4 treatment groups with 10 mice in each group. One group was treated with Compound (I) given by daily intraperitoneal injections (10 mg/kg/day) for 21 days. A second group received intravenous injections of paclitaxel (20 mg/kg) every 4 days for 3 doses. A third group received both paclitaxel and Compound (I) and a fourth group was given injections in the same way using vehicle alone. The experiments were replicated three times.

Results

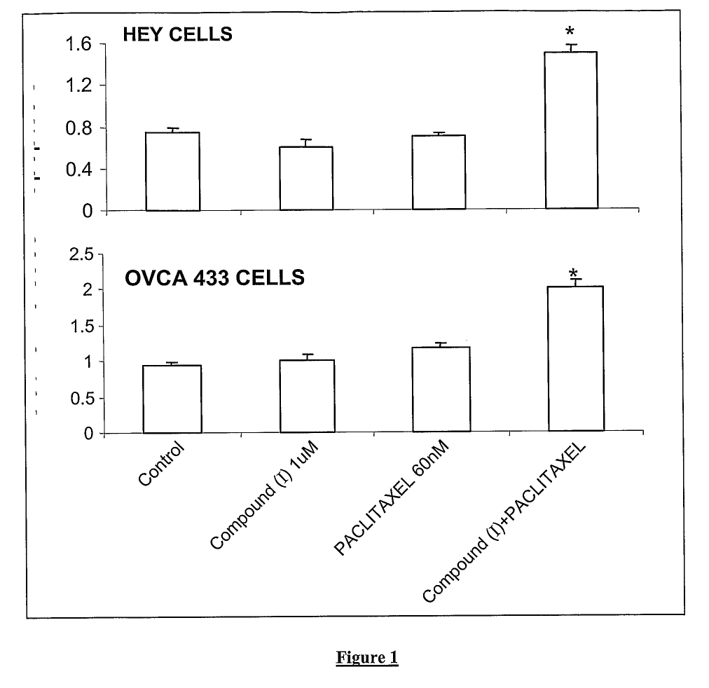

In vitro studies in ovarian cells: Addition of either Compound (I) or paclitaxel had no statistically significant effects on apoptosis. However, when Compound (I) was combined with paclitaxel there was a highly significant increase in apoptosis compared with vehicle treated control cells or either compound given alone. Results are shown in FIG. 1 where “*” indicates a statistically significant increase compared with controls.

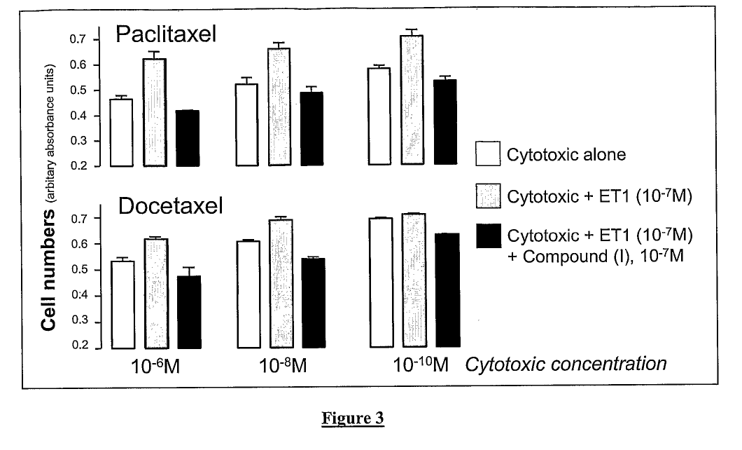

In vitro studies in prostate cells: Additional of increasing doses of either paclitaxel or docetaxel significantly reduced the numbers of viable prostate cells remaining in culture after 24 hours of treatment. This reduction in viable cell number was reversed by concomitant administration of endothelin-1, an effect which was blocked by Compound (I). See FIG. 3.

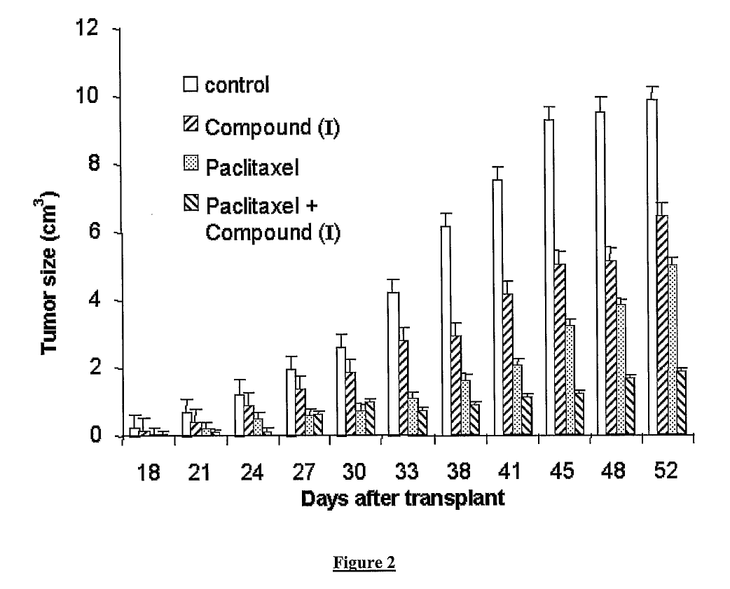

In vivo studies: Compound (I) as a monotherapy resulted in a significant inhibition of HEY ovarian cell xenografts. The degree of inhibition was similar to that achieved with paclitaxel given as monotherapy. The co-administration of Compound (I) with paclitaxel, caused a potentiating effect of Compound (I) on the anti-tumour effects of paclitaxel resulting in partial or complete tumour regression. Results are shown in FIG. 2.

Conclusions

These findings demonstrate that Compound (I), a specific endothelin receptor antagonist, potentiates the effects of paclitaxel on apoptosis in ovarian cells in vitro and the growth inhibitory properties of paclitaxel in ovarian tumours in vivo. Furthermore, compound (I) reverses the inhibitory effects of endothelin-1 on cytotoxic-induced (paclitaxel or docetaxel) cell death. Thus, Compound (I) in combination with paclitaxel or docetaxel is potentially useful in the treatment of cancers.

1-25. (canceled)

26. A combination, comprising N-(3-methoxy-5-methylpyrazin-2-yl)-2-(4-[1,3,4-oxadiazol-2-yl]phenyl)pyridine-3-sulphonamide and an anti-mitotic cytotoxic agent.

27. The combination according to claim 26 wherein the anti-mitotic cytotoxic agent is a taxane.

28. The combination according to claim 26 wherein the anti-mitotic cytotoxic agent is an epothilone.

29. The combination according to claim 26 wherein the anti-mitotic cytotoxic agent is an epothilone A derivative or analogue thereof.

30. The combination according to claim 26 wherein the anti-mitotic cytotoxic agent is an epothilone B derivative or analogue thereof.

31. The combination according to claim 26 wherein the anti-mitotic cytotoxic agent is an epothilone C derivative or analogue thereof.

32. The combination according to claim 26 wherein the anti-mitotic cytotoxic agent is an epothilone D derivative or analogue thereof.

33. The combination according to claim 26 wherein the anti-mitotic cytotoxic agent is a vinca alkaloid derivative or analogue thereof.

34. The combination according to claim 26 wherein the anti-mitotic cytotoxic agent is paclitaxel.

35. The combination according to claim 26 wherein the anti-mitotic cytotoxic agent is docetaxel.

36. The combination according to claim 26 wherein the anti-mitotic cytotoxic agent is ixabepilone.

37. The combination according to claim 26 wherein the anti-mitotic cytotoxic agent is patupilone.

38. The combination according to claim 26 wherein the anti-mitotic cytotoxic agent is vinorelbine.

39. The combination according to claim 26 wherein the anti-mitotic cytotoxic agent is XAA296.

40. The combination according to claim 26 wherein the anti-mitotic cytotoxic agent is T-138067.

41. A pharmaceutical composition which comprises a combination as claimed in claim 26 in association with one or more pharmaceutically acceptable diluents or carrier.

42. A method of treating cancer, in a warm-blooded animal, such as man, in need of such treatment which comprises administering to said animal an effective amount of a combination as claimed in claim 26.

43. The method according to claim 42 wherein said cancer is oesophageal cancer, myeloma, hepatocellular, pancreatic, cervical cancer, ewings tumour, neuroblastoma, kaposis sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, brain cancer, renal cancer, lymphoma or leukaemia.

44. The method according to claim 42 wherein said cancer is prostate cancer.

45. A kit comprising:
a) Compound (I), in a first unit dosage form;
b) an anti-mitotic cytotoxic agent; in a second unit dosage form; and
c) container means for containing said first and second dosage forms; and optionally
d) with instructions for use.
